(19) United States
(12) Patent Application Publication
(10) Pub. No.: US 2019/0269339 A1
(43) Pub. Date: Sep. 5, 2019

(54) IMPLANTS FOR MONITORING PHYSIOLOGICAL PARAMETERS WITHIN LIVING BODIES

(71) Applicant: Integrated Sensing Systems, Inc., Ypsilanti, MI (US)

(72) Inventor: Nader Najafi, Ann Arbor, MI (US)

(21) Appl. No.: 16/293,548

(22) Filed: Mar. 5, 2019

Related U.S. Application Data
(60) Provisional application No. 62/710,929, filed on Mar. 5, 2018.

(51) Int. Cl. A61B 5/0215; A61B 5/00

(52) U.S. Cl. CPC A61B 5/02158; A61B 5/0031; A61B 5/686; A61B 2560/063; A61B 5/0004; A61B 2560/0219; A61B 2562/182; A61B 5/6862

(57) ABSTRACT

Wireless implants and systems and methods that employ such implants to monitor multiple physiological parameters within living bodies, such as monitoring cardiovascular pressures/hemodynamics. Such an includes a hermetically-sealed housing having an elongate shape that defines oppositely-disposed first and second ends of the implant, first and second sensing elements located at the first and second ends of the implant, respectively, and at least one wireless transmitting device within an internal cavity of the implant and connected to the first and second sensing elements for wirelessly transmitting output signals generated by the first and second sensing elements. The implant is adapted to be implanted to monitor at least two different physiological parameters, for example, the pulmonary artery pressure and pulmonary capillary wedge pressure as two different cardiovascular pressures within the human body.

### CROSS-REFERENCE TO RELATED APPLICATIONS

This application claims the benefit of U.S. Provisional Application No. 62/710,929, filed Mar. 5, 2018, the contents of which are incorporated herein by reference.

### BACKGROUND OF THE INVENTION

The present invention generally relates to the field of implantable medical devices for monitoring physiological parameters. More particularly, the invention relates to a system and delivery method for monitoring cardiovascular pressures/hemodynamics using an implants equipped with pressure sensors, including sensors for monitoring the progression, treatment of, and long-term management of different chronic diseases such as but not limited to congestive heart failure (CHF), congenital heart diseases (CHD), pulmonary hypertension (PHP), cardiac structural heart diseases (SHD), patients with arrhythmia such as atrial fibrillation (A-fib) and atrial fluttering, patients in need of acute and ICU management, patients at risk for pericardial effusion, patients after a valvular replacement/repair, patients with hypertension, patients at risk for thromboembolic events, and other conditions of the cardiovascular system.

The cardiovascular system of a human (as well as other animals) includes a systemic circulation system and a pulmonary circulation system. The pulmonary circulation is a “loop” through the lungs where blood is oxygenated, and the systemic circulation is a loop through the rest of the body to provide oxygenated blood thereto. The systemic circulation can also be seen to function in two parts - a macro-circulation and a micro-circulation, with different blood vessels. The macro-circulation system includes arteries by which oxygenated blood enters the systemic circulation after leaving the left ventricle (LV) of the heart through the aortic semilunar valve, and veins which return the blood to the right atrium (RA) of the heart. The micro-circulation system can be viewed as including capillaries that receive blood flow from the arteries and through which blood flows to body tissues before returning to the heart through the veins.

The first part of the systemic circulation is the aorta, a massive and thick-walled artery. The aorta arches and gives branches supplying the upper part of the body. The aorta enters the abdomen after passing through the aortic opening of the diaphragm at the level of thoracic ten vertebra, and later descends to supply branches to the abdomen, pelvis, perineum, and lower limbs. The walls of the aorta are elastic, which helps to maintain the blood pressure throughout the body. When the aorta receives almost five liters of blood from the heart, it recoils and is responsible for pulsating blood pressure. Moreover, as the aorta branches into smaller arteries, their elasticity decreases and their compliance increases. Arteries branch into small passages called arterioles and then into the capillaries. After their passage through body tissues, capillaries merge once again into venules, which continue to merge into veins. The venous system finally coalesces into two major veins: the superior vena cava (roughly speaking, draining blood from areas above the heart) and the inferior vena cava (roughly speaking, receiving blood from areas below the heart). The superior and inferior vena cava empty into the right atrium of the heart. Additionally, coronary vessels supply blood to the heart as part of a small loop of the systemic circulation and derives very little from the blood contained within the four chambers.

Various conditions of the cardiovascular system can be diagnosed and monitored by sensing pressures within the heart, cardiovascular system, and coronary arteries. Another condition diagnosed and monitored by evaluating pulmonary artery (PA) pressure is primary pulmonary hypertension (PPH). In addition to direct invasive measurement using a catheterization procedure, Doppler echocardiography has been used as a method for evaluating PPH, though it too requires specialized equipment in a dedicated laboratory. The course of patients with PPH is usually long and chronic, and many treatment modalities have been proposed but none to date provide an absolute solution. Therefore, following diagnosis of pulmonary hypertension, it is desirable to noninvasively monitor this condition on a continuing basis in order to optimize treatment.

Congestive heart failure (CHF) is a condition in which a damaged or overworked heart cannot pump adequately to meet the metabolic demands of the body and/or can do so only with an elevated ventricular diastolic pressure. CHF is a major health problem worldwide, affecting millions of patients and accounts for numerous hospitalizations. Overall, the cost of treating CHF is very high (billions of U.S. dollars annually) and involves numerous physician visits. From 1979 to 1999, CHF deaths increased 145% and hospital discharges increased 155%. Survival is poor with 20% dying within one year and only 50% of patients surviving more than five years. The many patients suffering from this progressive, fatal disease tend to have an extremely poor quality of life and become increasingly unable to perform routine daily tasks. Another group involves a variety of congenital heart diseases (CHD).

Hemodynamic monitoring solutions for monitoring and managing main chronic cardiac diseases, such as CHF, CHD, PHP, arrhythmia, etc., are listed below.

Two independent (simultaneous or sequential) measurements of the two circulation systems (systemic and pulmonary circulation systems).

Measurements of pressures in the systematic circulation system: Left heart filling pressure (LHFP) (left ventricle (LV) or left atrium (LA)); and pulmonary capillary wedge pressure (PCWP).

Measurements of pressures in the pulmonary circulation system: Right atrium (RA); right ventricle (RV); and pulmonary artery (PA).

Left heart filling pressure (LHFP) is a key factor in the progression of CHF. Left heart filling pressure represents the diastolic pressure at which the left atrium (LA) and left ventricle (LV) equilibrate, at which time the mitral valve opens (or closes) and the left ventricle fills with blood from the left atrium. As the heart ages, cardiac tissue becomes less compliant, causing the left ventricle filling pressure to increase. This means that higher pressures are required from the left atrium in order to fill the left ventricle. The heart must compensate for this to maintain adequate cardiac output (CO). However, increasing the left atrium pressure strains the heart and over time irreversible alteration will occur.

The left heart filling pressure (LHFP) is among the primary factors physicians use to evaluate CHF patients. Left ventricular end diastolic pressure (LVEDP) and mean left atrium pressure (MLAP) correspond directly with left heart filling pressure and are easy for physicians to identify from left ventricle or left atrium pressure data. The physician's ultimate goal is to increase cardiac output while maintaining a reasonable left heart filling pressure. Treatment methods include medications, lifestyle changes, pacemakers, and/or surgery.

As with the above-noted CHF, CHD and PHP conditions, the current method for evaluating intracardiac pressures such as MLAP and LVEDP is an invasive cardiac catheterization procedure. In certain cases, CHF is complicated by mitral stenosis, necessitating significantly more precise and continuous pressure data. Atrial fibrillation (A-fib) can develop as a result of this condition, and the evaluation of such cases is considerably more complex since pressure gradients across the mitral valve must also be measured.

Compared to other cardiac locations, left heart medical devices and procedures provide additional challenges due to the extreme vulnerability of the left side of the heart to thrombi that can cause stroke. In fact, 20% of all strokes in the U.S. are related to the thrombi generated due to left-heart atrial fibrillation.

Instead of monitoring left heart (either left atrium and left ventricle), the diagnosis of left ventricle failure and mitral stenosis can be obtained by measuring the pulmonary capillary wedge pressure (PCWP), which provides an indirect measurement of MLAP (directly corresponding to left heart filling pressure). The current catheter procedure for measuring PCWP is to advance a balloon-tipped multi-lumen (e.g., Swan-Ganz) catheter through the right atrium (RA) and right ventricle (RV) until the distal tip of the catheter is located within a branch of the pulmonary artery. The balloon is then inflated to occlude the pulmonary artery branch, and a pressure transducer distal of the balloon measures the pressure within the pulmonary artery branch, which drops as a result of the occlusion and stabilizes at a pressure level approximately equal to MLAP. PCWP tracing approximates actual left atrium tracing but is slightly delayed since pressure wave is transmitted retrograde through pulmonary veins.

For monitoring systemic circulation, the measurement of PCWP offers the ability to collect the most useful information (equal to left heart monitoring for LHFP) and safety of right heart/PA medical devices and procedures.

Measurement of the pulmonary artery hemodynamic is inferior in most indications compared to PCWP. Monitoring mean pulmonary artery pressure (MPAP) provides a shadow of the left heart filling pressure (i.e., MLAP). As long as the shadow relationship between MPAP and MLAP is one-to-one, then using MPAP to manage cardiac medication titration to sustain a reasonable left-heart filling pressure would be productive. However, since there are many parameters that may change MPAP without a substantial change in the left-heart filling pressure (MLAP), the patients with pulmonary artery pressure (PAP) implants are potentially at risks of receiving the appropriate treatment. For example, if there is a pulmonary comorbidity, a physician could potentially relate the event to CHF related condition, which and may delay or prevent appropriate medical action. Another example, if a patient is going through a pulmonary embolism episode that changes MPAP without a substantial change in MLAP. In this event, the physician may potentially address a patient with CHF-related medication titration instead of addressing a pulmonary embolism event. These examples present the vulnerability of monitoring PAP as hemodynamic parameter in patients who have or are at the risk of developing pulmonary vascular diseases. Monitoring left atrium pressure (LAP) is a more critical parameter than PAP for the differentiation between pulmonary embolism and CHF exacerbation.

A more in-depth look at the cardiovascular system may shed more lights on these two important risk factors. The pulmonary artery (PA) and the left atrium (LA) are connected via a hydraulic (blood) path and their relationship is determined by laws of fluidic dynamics, which can be generally characterized as follows:

PAP=LAP+(Blood Flow Rate)x(Fluidic Resistance between the LA and PA)

This connecting fluidic path includes the pulmonary arteries (PA), the pulmonary artery capillaries, the pulmonary vein capillaries, and the pulmonary vein. The pulmonary path is also affected by lung mechanical properties and airway pressures. A change in any element of this blood path can change PA pressure similar to that of an LA change without an actual LA change. In a healthy pulmonary system, PA pressure correlates with LA pressure, and thus maintaining a fixed MPAP often results in maintaining a fixed MLAP (left heart filling pressure).

Comorbidities and problems with lung or pulmonary blood vessels adversely affect the correlation between LAP and PAP. When PA pressure changes, it is very challenging to know whether CHF caused the change or the comorbidity and thus the associated patients were exposed to unnecessary additional risks. Here, maintaining a fixed MPAP may not result in maintaining a fixed MLAP (i.e., left heart filling pressure) and thus an incorrect medication may be prescribed.

In the last decade, new developments of wireless implantable hemodynamic monitors (IHM) have become available, including implants delivered by a catheter and placed in the atrial septum to measure LA pressures/hemodynamics and implants delivered by a catheter and placed in the PA to measure PCWP or PA pressures/hemodynamics (U.S. Pat. No. 8,512,252), both available from Integrated Sensing Systems Incorporated (ISS). Other existing technologies generally relate to monitoring either LA, LV, or PA.

### BRIEF SUMMARY OF THE INVENTION

The present invention provides wireless implants and systems and methods that employ such implants to monitor multiple physiological parameters within living bodies, such as monitoring cardiovascular pressures/hemodynamics.

According to one nonlimiting aspect of the invention, a wireless implant is provided that includes a hermetically-sealed housing having an elongate shape that defines oppositely-disposed first and second ends of the implant, first and second sensing elements located at the first and second ends of the implant, respectively, and wireless transmitting means within an internal cavity of the implant and connected to the first and second sensing elements for wirelessly transmitting output signals generated by the first and second sensing elements. The implant is adapted to be implanted to monitor at least two different physiological parameters, for example, the pulmonary artery pressure and pulmonary capillary wedge pressure as two different cardiovascular pressures within the human body.

Other nonlimiting aspects of the invention include various different methods of implanting the implant described above in a living body.

Other aspects and advantages of this invention will be appreciated from the following detailed description.

### BRIEF DESCRIPTION OF THE SEVERAL VIEWS OF THE DRAWING

FIGS. 1, 2 and 3 each schematically represent an implant that contains two wireless pressure sensors each adapted to independently monitor a different physiological parameter within a living body in accordance with nonlimiting embodiments of the invention.

FIGS. 4A through 4F and 5 each schematically represent embodiments in which an implant comprises two housings, each containing a wireless pressure sensor, wherein the housing are coupled to enable the sensors to monitor two different physiological parameters within a living body in accordance with nonlimiting embodiments of the invention.

FIGS. 6, 7, and 8 each schematically represent embodiments of implants similar to that represented in FIG. 3, and different modes for placing and anchoring the implant in a living body to monitor two different physiological parameters in accordance with nonlimiting embodiments of the invention.

### DETAILED DESCRIPTION OF THE INVENTION

The following disclosure will describe certain nonlimiting aspects of the invention in reference to wireless pressure sensor technologies and implantation procedures, including but not limited to sensor technologies and implantation procedures disclosed in U.S. Pat. No. 8,512,252, whose contents are incorporated herein by reference. U.S. Pat. No. 8,512,252 describes a wireless system for monitoring either PCWP (pulmonary capillary wedge pressure) or PA (pulmonary artery) pressure with an implanted pressure sensor, depending on the position of the pressure sensing membrane of the sensor relative to the delivery catheter or the pulmonary artery. If the pressure sensing membrane faces the distal end of the delivery catheter, then it measures PCWP; otherwise it measures PA pressure.

As previously discussed, as a single cardiac pressure measurement system, the measurement of PCWP is useful for many indications: the most useful information (equal to left heart monitoring) and the high level of safety of right heart/PA medical devices/procedures. However, it has been observed that after the implanted pressure sensor is fixed in a pulmonary artery vessel having an appropriately-sized diameter, it is possible that the pressure on one side of the sensor may interfere with the pressure the other side (for example, PCWP interferes with the sensing of PA pressure) due to the fact that the implant has not completely blocked the pulmonary artery in which it is implanted, even after some cell growth has occurred overtime. As a solution, embodiments disclosed herein provide implants as part of an IHM system capable of improving the measurement of two different pressures. As such, embodiments depicted in the drawings represent configurations of implants that are particularly well suited to promote a more optimized hemodynamic monitoring system to assist medical staff to better manage chronic cardiac diseases by enabling the simultaneous, sequential, or independent monitoring of two sets of measurements, particular but nonlimiting examples of which are PA pressure and PCWP, to provide for a practical monitoring technique employed in the systemic and pulmonary circulations systems. As a matter of convenience, the following discussion will make reference to measuring PA pressure and PCWP, though it should be evident that other pressures could be sensed. For convenience, consistent reference numbers are used throughout the drawings to identify the same or functionally related or equivalent elements.

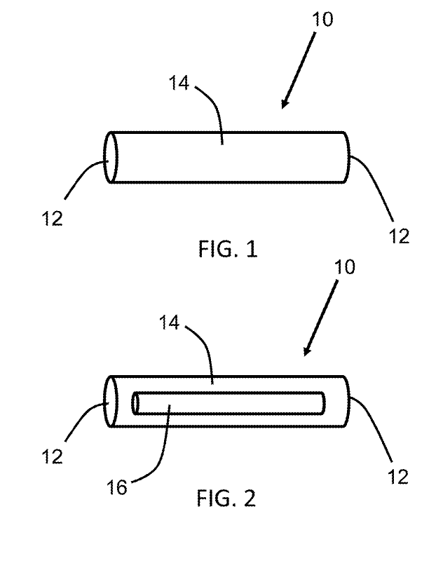

FIG. 1 represents a nonlimiting embodiment of the invention in which a single implant 10 is adapted to measuring both PA pressure and PCWP. The implant 10 has two sensing elements 12, one at each end of a hermetically-sealed housing 14 of the implant 10. The housing 14 is referred to herein as elongate because its length is at least several times greater than its width. Preferably the dual-sensing implant 10 is cylindrical-shaped and the two pressure elements 12 are placed at the longitudinally-opposite flat ends of the implant 10. It is foreseeable that the implant 10 could be configured to have other shapes for the housing 14 and other placements for the sensing elements 12. The dual-sensing implant 10 can be sized and shaped to be placed in the pulmonary artery so that one sensing element 12 is positioned to measure PCWP and the other sensing element 12 measures PA pressure. When placed with a catheter, the sensing element 12 positioned to measure PCWP would be the distal end of the implant 10 and the sensing element 12 positioned to measure PA pressure would be the proximal end of the implant 10 relative to the implantation procedure.

Each sensing element 12 is a transducer, preferably a MEMS device and more particularly a micromachine fabricated by additive and subtractive processes performed on a substrate. The substrate can be rigid, flexible, or a combination of rigid and flexible materials. Notable examples of rigid substrate materials include glass, semiconductors, silicon, ceramics, carbides, metals, hard polymers, and TEFLON. Notable flexible substrate materials include various polymers such as parylene and silicone, or other biocompatible flexible materials. A particular but nonlimiting example of a suitable transducer for hemodynamic monitoring of various blood pressures within the cardiovascular system is a MEMS capacitive pressure sensor for sensing pressure, though other materials and any variety of sensing elements, e.g., capacitive, inductive, resistive, piezoelectric, etc., could be used. Additionally, the implant 10 may include one or more additional transducers configured to sense temperature, flow, acceleration, vibration, pH, conductivity, dielectric constant, and chemical composition, including the composition and/or contents of a biological fluid, for example, oxygen, carbon dioxide, glucose, gene, hormone, or gas content of the fluid.

As represented in FIG. 2, the implant 10 preferably further includes one or more internal components 16, as nonlimiting examples, electronic circuitry, for example, an application specific integrated circuit (ASIC). The electronic circuitry may operate so that measurements performed or output by the sensing elements 12 occur simultaneously, sequentially, or independently. The electronic circuitry operates in combination with an antenna to wirelessly transmit and receive data and other communications to a remote device, such as a readout unit, which may also tele-power the implant 10. The wireless operation (tele-communication and/or tele-powering) of the implant 10 can be accomplished by different means, known to those skilled in the art, such as magnetic telemetry, RF telemetry, ultrasound telemetry, analog, digital, hybrid, mixed signal, etc. The antenna may comprise a coil (e.g., copper windings) wrapped around a core (e.g., ferrite), though other antenna configurations and materials are foreseeable. The internal components 16 of the implant 10 may further include a battery or other power storage device for powering the sensing elements 12 and other electronic components of the implant 10, but in preferred embodiments is powered entirely by a remote device (not shown) that is not configured for implantation, such as a readout unit. Such a readout unit may be configured to receive outputs from the sensing elements 12, process the signals, and relay the processed signals as data in a useful form to a medical clinician. Because the implant 10 is equipped with a built-in antenna, the implant 10 does not require a wire, cable, tether, or other physical component that conducts the outputs of the sensing elements 12 to a separate location where another component utilizes the outputs of the sensing elements 12 and/or transmits the outputs of the sensing elements 12 to a location outside the body of a patient. Though batteryless operation is preferred, the implant 10 may be configured for batteryless operation while its housing 14 also contains one or more batteries for continuously powering its sensing elements 12 and/or some of its internal components 16.

The two sensing elements 12 of the implant 10 can share one or more of the internal components 16. For example, the implant 10 may have a single antenna that is shared by the two sensing elements 12, each having separate processing electronics. Furthermore, the antenna may comprise a single ferrite core and two separate coils, one for each sensing element 12 and its electronics.

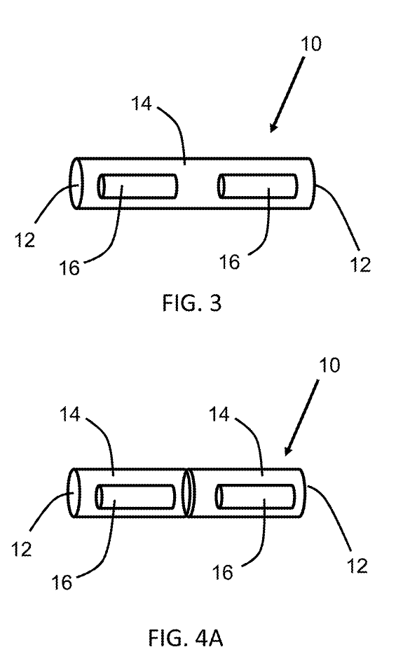

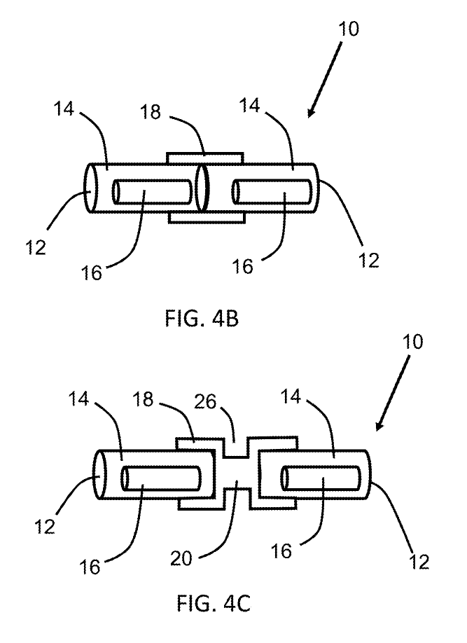

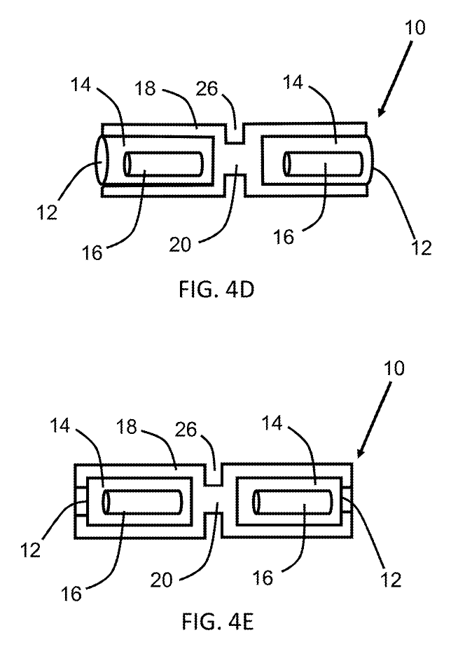

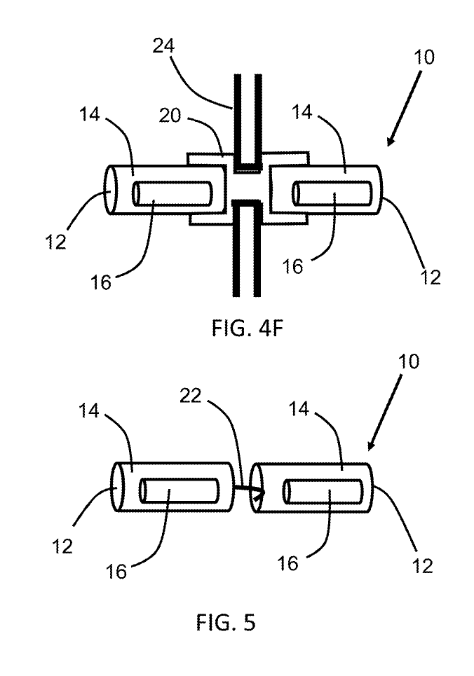

Alternatively, the sensing elements 12 may have entirely separate and independent internal components 16 within the housing 14, as is schematically represented in FIG. 3. The longitudinal distance between the two sets of internal components 16 may be selected to provide a longitudinal gap between the antennae to optimize the length of the implant 10 and its wireless tele-powering and tele-communication ranges.

Whereas FIGS. 1, 2, and 3 represent implants 10 whose sensing elements 12 and internal components 16 share a single hermetically-sealed housing 14, FIGS. 4A, 4B, 4C, 4D, 4E, 4F, and 5 represent implants 10 that each comprise two independent hermetically-sealed housings 14, each housing one of the two sensing elements 12. In FIG. 4A, the housings 14 are represented as being cylindrically-shaped and rigidly joined end to end to retain the overall cylindrical shape depicted for the implants of FIGS. 1, 2, and 3. FIG. 4B represents two cylindrically-shaped housings 14 rigidly held to abut end to end with a wrap or sleeve 18 that surrounds the abutting ends of the housings 14. In FIG. 4C, two cylindrically-shaped housings 14 are represented as being rigidly secured to each other with a sleeve 18 that includes a central spacer portion 20 that axially spaces the adjacent ends of the housings 14 apart. The sleeve 18 and its spacer portion 20 cooperate to define an external surface feature 26, represented as a recess, to facilitate the assembly of the implant 10 with an anchor, for example, such as an anchor 24 represented in FIG. 4F. Though represented as a recess in the form of a single circumferential groove, the feature 26 could be in the form of one or more protrusions, for example, knobs, bumps, or shoulders that project from the outer surface of the sleeve 18.

Whereas the sleeve 18 covers only portions of the longitudinal length of each housing 14 in FIGS. 4B and 4C, FIGS. 4D and 4E represent further embodiments in which the sleeve 18 covers the entire longitudinal length of each housing 14. Additionally, in the embodiment of FIG. 4E the sleeve 18 further covers at least a portion of each end of the implant 10, leaving exposed a portion of an end surface of each housing 14 so that the sleeve 18 does not interfere with the sensitivity or sensing capability of their associated sensing element 12.

In FIG. 5, two cylindrically-shaped housings 14 are represented as being secured to each other with a tether 22 that spaces the housings 14 apart but allows for some off-axis movement of the housings 14 relative to each other. The sleeve 18 of FIG. 4B and the spacer 20 of FIG. 4C may provide for a rigid or flexible coupling of the housings 14, the latter of which allows the implant 10 to be flexible at the connection between the housings 14, which facilitates movement of the dual-sensing implant 10 in a delivery catheter when curved passageways are encountered. Any of these attachment techniques can make use of flexible glues, polymers, or epoxies, hard glues, polymers, or epoxies, an anchor to couple the housings 14 (e.g., PEEK or NiTi anchors), tethers, and combinations thereof.

The output signals derived from the sensing elements 12 may be superimposed (modulated) on a carrier frequency by analog schemes and then read by an external readout unit, or superimposed on a carrier frequency by digital schemes and then read by an external readout unit, or a combination thereof. As previously noted, measurements performed by the sensing elements 12 and/or the associated outputs transmitted by the implant 10 for the elements 12 may occur simultaneously, sequentially, or independently.

The implants 10 of FIGS. 1 through 5 can be sized and shaped for placement in a single pulmonary artery vessel within the pulmonary artery system to sense two different pressures, most notably PCWP and PA pressure, at the opposite ends of the implant 10. As disclosed in U.S. Pat. No. 8,512,252, the implant 10 can be delivered to a large-diameter pulmonary artery and released so that the implant 10 is placed and becomes physically fixed in a smaller downstream pulmonary artery as a result of blood flow. Though a single implant 10 with multiple sensing elements 12 and optionally multiple housings 14 are preferred, it is foreseeable that the ability to sense two different pressures could be performed with two separate implants 10, each delivered and placed independently in the pulmonary artery system to independently and separately measure PA pressure and PCWP. The two implants 10 could be placed in the same pulmonary artery vessel or at different places within the pulmonary artery system.

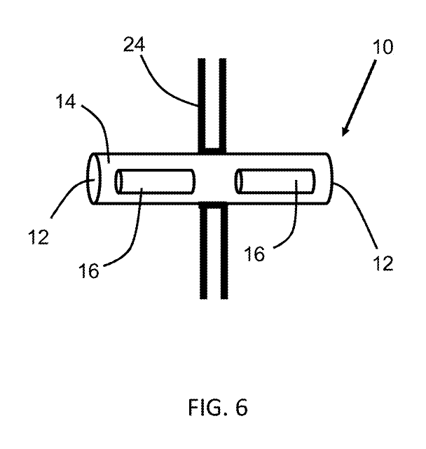

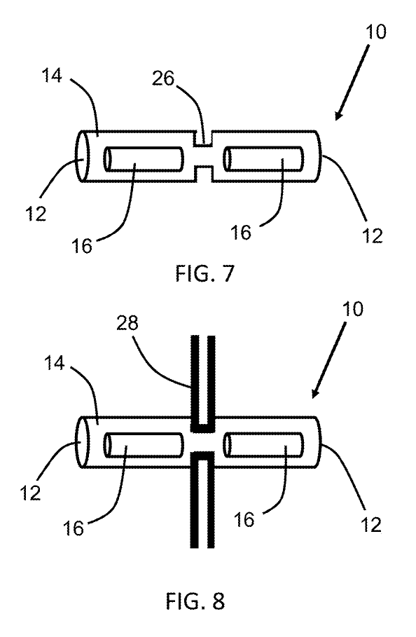

FIGS. 6, 7, and 8 represent implants 10 of the type represented in FIG. 3 adapted for placement in a septum (not shown), for example, the atrial septum (LA-RA) to monitor both left and right atrium pressures or the ventricle septum (LV-RV) to monitor both left and right ventricle pressures. In particular, the implant 10 is shown in FIG. 6 as being equipped with a septal anchor 24 to orient and position the implant 10 within the atrial septum to place one of its sensing elements 12 in the left atrium and the other sensing element 12 in the right atrium. Similar to the implants 10 of FIGS. 4C through 4F, the housing 14 of the implant 10 shown in FIGS. 7 and 8 is modified to have one or more external surface features 26 to facilitate the assembly of the implant 10 with an anchor, in this case, a septal anchor 28. Though represented as two individual recesses, the features 26 could be provided by a single circumferential groove or be in the form of one or more protrusions, for example, knobs, bumps, or shoulders that project from the outer surface of the housing 14. Similar placement of an anchor is provided for with the implant depicted in FIG. 3. In the embodiment represented in FIGS. 3, 6, 7, and 8, two antennae (e.g., two separate sets of ferrite cores and coils) are spaced apart along the length of the implant 10 for placement of an anchor surrounding the longitudinal gap between them to yield an implant configuration with a balanced weight distribution.

In addition to the septal anchors 24 and 28 schematically represented in FIGS. 6 and 8, other anchoring devices suitable for use with the implants 10 can be similar to those used as vascular closure devices, atrial septum defect occluder devices (ASD and PFO (patent foramen ovale) occluders), and closure paravalvular leak devices. Nonlimiting examples of such devices include the CELT ACD® produced by Vasorum Ltd. or various devices produced by Occlutech International AB. The use of anchors adapted to place the implants 10 in the walls of various other organs is also foreseeable.

In addition to monitoring pressures, the implants 10 can be used as acoustic sensors to measure the health of the cardiovascular system or monitor other implanted devices, such as ventricular assist devices (VADs) including ventricular assist devices (LVAD).

Embodiments of the implants 10 described above are also capable of being attached to or used in combination with one or more implantable medical devices, such as a wireless implantable sensor/stimulator, implantable medical devices including but not limited to stents (as nonlimiting examples, vein stents, pulmonary stents, coronary stents, intracranial stents, etc.), vascular closure devices, atrial septum defect occluder devices (including ASD and PFO occluders), closure paravalvular leak devices, AFR (atrial flow regulator) devices, vascular reconstruction devices, heart valves, heart valve repair products, and left atrium appendage occluders and closure devices. Examples of such medical devices are commercially available from various manufacturers, including but not limited to Vasorum, Occlutech, Hubless, Boston Scientific, Abbott, Cardia, Gore Medical, Microport, Lepu Medical, Medtronic, and Biotronic.

Embodiments of the implants 10 described above are further capable of being attached to or comprise one or more add-on features (not shown) for enabling or facilitating the coupling of the implants 10 to an anchor, stent, LAA occlude, vascular closure device/anchor, or any other type of closure/anchoring device. Such add-on features may be utilized to reduce the stress of the attachment of a metallic anchor to the implant 10. Such add-on features are also preferably configured so that the portions of the implant 10 containing the sensing elements 12 and any antenna (or antennae), or at least the coil of any antenna, will be located within the organ being monitored by the implant 10 in order to avoid the Faraday-cage effect. For this reason, an add-on feature preferably places a metallic anchor or other metallic component a suitable distance from any antenna within the implant 10. Preferred add-ons have little or no impact on the diameter of the delivery apparatus used to deliver the implant 10. Suitable add-ons may be integrally formed with any part of the implant 10 or added to the implant 10 after its assembly (e.g., housing 14, sensing elements 12, and internal components 16), and may take a variety of different forms, including features that are external and/or internal (e.g., cavities) of the implant 10.

Embodiments of the implants 10 represented in FIGS. 1 through 8 are capable of achieving a reliable seal between the housing 14 thereof and the walls of the pulmonary artery and the atrial and ventricle septa. This capability can be promoted by modifications to the implant housing 14, including but not limited to any one or more of the following adapted to promote or assist with sealing: application of a cell-growth promoting material to the implant housing 14 to promote faster and thicker cell growth to seal the pulmonary artery (preferably with no additional cell growth promoted over the surfaces of the sensing elements 12); one or more features on the exterior of the implant housing 14, such as bumps, recesses, corrugations, and/or rough surfaces, to promote better adhesion to the pulmonary artery wall; one or more anchors of a selected material (e.g., one or more of polymer, metallic, shape memory alloy, and steel metals) or cell growth-promoting coating; or one or more anchors having one or more features, such as bumps, recesses, corrugations, and/or rough surfaces, to promote better adhesion to the pulmonary artery wall. The implants 10 can also be assembled with one or more anchors configured to dilate the pulmonary artery to allow the implant 10 to be placed in a pulmonary artery vessel with a relatively small diameter.

Other steps can be additionally or alternatively performed during the implantation procedure to temporarily expand/enlarge blood vessels during implantation to allow the implant 10 to be placed in a pulmonary artery vessel with a relatively small diameter, in particular, a vessel having a diameter smaller than the diameter of the implant 10. The temporary expansion of the pulmonary artery vessels can be accomplished by different means, including but not limited to exposing the patient to one or more liquid or gaseous drugs or chemicals, as a nonlimiting example, nitrous oxide.

Delivery of the IHM implants 10 can be performed in different ways, including but not limited to one or more of percutaneously, minimally invasive, and catheter delivery. Suitable catheter delivery approaches include but are not limited to through the femoral vein (similar to a PCWP catheterization or right heart catheterization or Swan-Ganz catheterization process), processes similar to placing a central venous catheter (CVC), also known as a central line, central venous line, or central venous access catheter. More specifically, the implants 10 can be delivered with a catheter placed in the veins in the neck (internal jugular vein), chest (subclavian vein or axillary vein), or through veins in the arms (also known as peripherally inserted central catheter (PICC) line). Minimally invasive and percutaneous approaches can also make use of the above-mentioned entry points or any other suitable point.

Conventional IHM systems demand both a medical facility with sophisticated equipment and highly specialized medical experts (e.g., interventional cardiologists, heart surgeons, or both) to place an IHM implant. As an example, conventional catheter-delivered IHMs are implanted in a heart catheterization facility by a full staff of interventional cardiology, while conventional surgical IHM implants are placed in a heart surgery facility by a cardiac surgeon. Conventional IHM implants placed via minimally invasive surgery can be implanted in a catheterization, surgery, or hybrid facility and also demands specialists. However, the aforementioned IHM implants cannot be implanted in standard emergency rooms, ICU units, or critical care units by other medical experts such as emergency physicians, critical care staff, ICU staff, or anesthesiologists.

In contrast, the IHM implants 10 disclosed herein can be implanted in patients located in facilities that have less sophisticated medical equipment than a heart catheterization lab, heart surgery room, or hybrid room, and by medical staff other than interventional cardiologists and cardiac/vascular surgeons. The IHM implants 10 disclosed herein can be implanted in emergency rooms, ICU units, critical care facilities, and even in specialized physician offices. For example, as disclosed in U.S. Pat. No. 8,512,252, a critical care specialist can deliver the implant 10 to a large-diameter pulmonary artery and release the implant 10 for automatic placement in a downstream pulmonary artery as a result of blood flow. Such an implantation process is similar to a standard central venous catheters (CVC) procedure in which a catheter is placed into a large vein, for example, in the neck (internal jugular vein), chest (subclavian vein or axillary vein), groin (femoral vein), or through veins in the arms (PICC line). The internal jugular vein is a particularly suitable site for IHM implantation since it is a preferred venous access for other large medical devices. Internal jugular venous access (especially right-sided) is associated with a low rate of catheter malposition, and is commonly used in situations that require reliable tip positioning for immediate use, such as drug administration or transvenous pacing. Similarly, the direct route from the right internal jugular vein to the superior vena cava facilitates hemodialysis access and pulmonary artery catheter placement. IHM implantation through veins in the arms similar to that of a PICC process is also believed to be particularly suitable.

The implants 10 described herein may also be released in one of the following locations: main pulmonary artery, smaller pulmonary artery than lumbar pulmonary artery but larger than the diameter of the implant, a pulmonary artery with a similar diameter as the implant, a pulmonary artery with a smaller diameter than the implant, right after the pulmonary valve, or before the pulmonary valve. In a simple but effective approach for delivery of one of the implants 10, an off-the-shelf or custom-made thin braided catheter can be used. In a manner similar to a right heart catheterization (RHC), the catheter can be placed in a pulmonary artery (preferably lumbar pulmonary artery) and the implant 10 released either by a pusher or by hydraulic force (pushing saline through the catheter). Blood flow will take the implant 10 to a pulmonary artery of appropriate size for the implant 10. An advantage of the dual-sensor implants 10 described above is that if the implant 10 were to flip (rotate end over end) during the delivery, it will still be capable of measuring both PA pressure and PCWP due to its longitudinal symmetry.

Particularly suitable methods for delivering one of the implants 10 are believed to entail attaching/suturing the proximal end of the implant 10 in a blood vessel at close proximity to the heart so that the distal end of the implant 10 measures physiological parameters associated with the heart (e.g., pressure, temperature, hemodynamic, 02 level, etc.). In particular, in order to monitor left atrium pressure (LA), the implant 10 is preferably placed in the pulmonary vein. The right superior pulmonary vein (RSPV) is believed to be preferred because in most cardiac surgeries there is an incision to place an LA succession/vent catheter as part of the normal operation. The same incision in the RSPV can be used to secure the implant 10. Other blood vessels can be used for different heart chambers. At the end of open chest surgery, the implant 10 can be placed through an incision in the border between the LA and the right superior pulmonary vein used as part of routine care for placement of an LV vent. The proximal end of the implant 10 can be sutured to the wall of the right superior pulmonary vein. Implant positioning, free from the wall of the pulmonary vein or atrium, can be confirmed with echocardiogram.

While the invention has been described in terms of particular embodiments, it should be apparent that alternatives could be adopted by one skilled in the art. As such, it should be understood that the above detailed description is intended to describe the particular embodiments and certain but not necessarily all features and aspects thereof, and to identify certain but not necessarily all alternatives to the embodiments and described features and aspects. As a nonlimiting example, the invention encompasses additional or alternative embodiments in which one or more features or aspects of the disclosed embodiments could be eliminated or two or more features or aspects of different embodiments could be combined. Accordingly, it should be understood that the invention is not necessarily limited to any embodiment described herein or illustrated in the drawing, and the phraseology and terminology employed above are for the purpose of describing the disclosed embodiment and do not necessarily serve as limitations to the scope of the invention. Therefore, the scope of the invention is to be limited only by the following claims.

1. An implant for monitoring two separate physiological parameters within a living body, the implant comprising:
a hermetically-sealed housing having an elongate shape that defines oppositely-disposed first and second ends of the implant;
first and second sensing elements located at the first and second ends of the implant, respectively; and
wireless transmitting means within an internal cavity of the implant and connected to the first and second sensing elements for wirelessly transmitting output signals generated by the first and second sensing elements.

2. The implant of claim 1, wherein the physiological parameters are two different cardiovascular pressures.

3. The implant of claim 2, wherein the two different cardiovascular pressures are pulmonary artery pressure and pulmonary capillary wedge pressure.

4. The implant of claim 2, wherein the two different cardiovascular pressures are left and right atrial pressures.

5. The implant of claim 2, wherein the two different cardiovascular pressures are left and right ventricle pressures.

6. The implant of claim 1, wherein the wireless transmitting means comprises a single antenna connected to each of the first and second sensing elements.

7. The implant of claim 1, wherein the wireless transmitting means comprises first and second antennae connected to the first and second sensing elements, respectively.

8. The implant of claim 7, wherein the first and second antennae are longitudinally spaced apart within the implant.

9. The implant of claim 1, wherein the housing is a hermetically-sealed first housing that contains the first sensing element, the implant comprises a hermetically-sealed second housing that contains the second sensing element, and the wireless transmitting means comprises first and second antennae located within the first and second housings, respectively, and connected to the first and second sensing elements, respectively.

10. The implant of claim 9, wherein the first and second housings are joined and abutting end to end.

11. The implant of claim 9, wherein the first and second housings are abutting end to end and joined together by a sleeve.

12. The implant of claim 11, wherein the sleeve entirely covers the implant except for at least a portion of each of the first and second ends of the implant.

13. The implant of claim 9, wherein the first and second housings are coupled together and arranged end to end with a sleeve comprising a spacer portion between the first and second housings such that the first and second housings do not abut end to end.

14. The implant of claim 9, wherein the first and second housings are coupled together and arranged end to end with a tether between the first and second housings.

15. The implant of claim 1, further comprising anchoring means for securing the implant within a living body.

16. The implant of claim 15, wherein the wireless transmitting means comprises first and second antennae connected to the first and second sensing elements, respectively, the first and second antennae are longitudinally spaced apart within the implant, and the anchoring means is disposed around the implant and surrounds a gap between the first and second antennae.

17. The implant of claim 15, wherein the anchoring means is a septal anchor.

18. The implant of claim 1, further comprising means associated with the implant to promote a seal between the implant and a wall of an internal organ, the promoting means being one or more of: a cell growth-promoting coating on surfaces of the implant; bumps, recesses, corrugations, and/or rough surfaces on an exterior of the implant; an anchor having a cell growth-promoting coating on surfaces thereof; an anchor having bumps, recesses, corrugations, and/or rough surfaces on an exterior surface thereof; and means for temporarily expanding a blood vessel.

19. The implant of claim 1, wherein the wireless transmitting means wirelessly transmits power to the implant.

20. The implant of claim 1, wherein the implant is attached to or used in combination with at least one of a stent, a vascular closure device, an atrial septum defect occluder device, a closure paravalvular leak device, an atrial flow regulator device, a vascular reconstruction device, a heart valve, a heart valve repair product, a left atrium appendage occluder, and a left atrium appendage closure devices.

21. The implant of claim 1, wherein the implant comprises at least one add-on feature that enables or facilitates coupling of the implant to at least one of an anchor, a stent, an LAA occlude, and a vascular closure device/anchor.

22. The implant of claim 21, wherein the add-on feature is positioned apart from the wireless transmitting means to avoid the Faraday-cage effect.

23. The implant of claim 21, wherein the add-on feature is configured to reduce stress due to attachment of an anchor to the implant.

24. A method of implanting the implant of claim 1 in a living body, the method comprising releasing the implant in the cardiovascular system of the living body to cause blood flow to deliver the implant to a pulmonary artery vessel so that the first and second sensing elements of the implant measure pulmonary artery pressure and pulmonary capillary wedge pressure as two different cardiovascular pressures within the human body.

25. A method of implanting the implant of claim 1 in a living body, the method comprising securing the implant in a wall of a vessel of the cardiovascular system of the living body.

26. The method of claim 25, wherein the vessel is a pulmonary artery.

27. A method of implanting the implant of claim 1 in a living body, the method comprising temporarily expanding a blood vessel during implantation of the implant to place the implant in a vessel having a diameter smaller than the implant.

28. The method of claim 27, wherein the blood vessel is temporarily expanded by exposing the living body to one or more liquid or gaseous drugs or chemicals.
